(19) United States
(12) Patent Application Publication
(10) Pub. No.: US 2022/0031523 A1
(43) Pub. Date: Feb. 3, 2022

(54) Athletic Apparel Configured to Manage Symptoms of Athletic Stress Induced Incontinence

(71) Applicant: Prickly Pear, LLC, Boulder, CO (US)

(72) Inventor: Kathleen L. Pierpoint, Boulder, CO (US)

(21) Appl. No.: 17/388,399

(22) Filed: Jul. 29, 2021

Related U.S. Application Data
(60) Provisional application No. 63/058,021, filed on Jul. 29, 2020.

(51) Int. Cl. A61F 13/47; A61F 13/514; A61F 13/537

(52) U.S. Cl. CPC A61F 13/4702; A61F 13/51456; A61F 2013/53035; A61F 13/53743; A61F 13/53717

(57) ABSTRACT

The invention described herein is directed to athletic apparel having a novel integrated gusset structure positioned in the apparel's crotch and further configured to capture and retain bodily fluids, and preferably uncontrolled urine released during athletic stress.

### CROSS REFERENCES TO RELATED APPLICATION

This application claims the benefit of and priority to U.S. Provisional Application No. 63/058,021, filed Jul. 29, 2020. The entire specification and figures of the above-referenced application is hereby incorporated in its entirety by reference.

### TECHNICAL FIELD

The invention generally relates to the field of garments, and more particularly, embodiments of the present invention relate to improved athletic apparel having a novel modular gusset configured to capture bodily fluids more efficiently.

### BACKGROUND

Millions of people experience involuntary body fluid excretion, including urine, menstrual fluid, vaginal discharge, sweating, and breast milk. In the case of involuntary excretion of urine, people with mild to moderate incontinence, which causes the release of 0.5 to more than 2.5 ounces of liquid urine in a single instance, have access to an abundance of products to solve these problems. For example, there are many disposable and non-disposable absorbent pads, diapers, and liners on the market today that are designed to absorb more than 0.5 ounces of liquid urine and keep the user of the product and their clothing dry. While these products provide the necessary protection for people with incontinence, for the reasons set out below, they are not a suitable solution for millions of people who experience minor and occasional leaks and drops of fluid, for example, caused by athletic stress induced incontinence

It has been estimated that as many as 1 in 3 women experience occasional drips and leaks of urine amounting to less than a teaspoon of urine in a single instance and would benefit from additional protection beyond that provided by the average undergarment. This problem is especially pronounced in older women, or women who have undergone childbirth, and those that actively participate in rigorous exercise, such as running or biking. Such minor excretions (the typical leak or drip is only 0.5 tsp. or less) do not require the high level of protection provided by the incontinence products of the prior art. Indeed, due to the numerous layers of such products, and in particular, the thick core that provides the absorption component, these products are bulky and uncomfortable to wear and would not be suitable, in particular for athletic apparel used for exercise. In many of the traditional products, the absorbent component is configured to expand as it receives and traps urine. Indeed, some pads or diapers will grow to be more than twice their original thickness.

In addition to the foregoing drawbacks of the products of the prior art because the products are constructed to accommodate bulky absorbent components and the weight attributable to entrapped leaked urine, the products do not allow wearers to comfortably wear low-coverage undergarments, which are common to athletic apparel generally. Absorbent pads must be placed into large, maximum-coverage undergarments that are capable of sufficiently containing them, while diapers and absorbent undergarments are likewise uniformly large in order to adequately address the incontinence of the wearer. Thus, a woman who prefers low-profile athletic apparel for exercising, or merely for comfort, is often forced to choose between either wearing her preferred underwear, which would risk leakage to her outer clothing, or wearing a cumbersome and unattractive garment that would ensure that all leaks are prevented. Finally, because the products of the prior art are generally made out of synthetic materials and are designed to trap excreted fluid such that it cannot escape, the ability for air to travel through such products is severely hindered. The lack of breathability can cause moisture to accumulate on the body of the wearer that does not pass through to the absorbent core, leading to discomfort, chafing, unpleasant odors, and the accumulation of bacteria leading to infection.

Because the available incontinence products are unsuitable for treating minor excretions, women experiencing these small drips and leaks of urine often choose to wear a disposable panty liner in their undergarments, despite the fact that such liners are generally intended to address menstrual fluid. While these liners offer some additional protection over the undergarment alone, the protection afforded generally does not rise to the level required by a wearer experiencing leaks of between approximately 0.25 tsp to approximately 0.75 tsp occasionally throughout the day. Nor are such a disposable panty liner always suitable for use in extended exercise situations, such as a marathon or other high intensity training. As with the incontinence products, these liners will reach capacity as they trap more and more leaks over time, causing the wearer to experience the wetness and discomfort associated with a saturated liner. Thus, in order to use these liners comfortably, it is necessary to periodically change out a used liner for a fresh one, an inconvenient and wasteful practice, that again can be impractical during prolonged exercise session or even athletic competitions. Moreover, liners, like the thicker pads and incontinence undergarment products, will not offer adequate breathability to the wearer, increasing discomfort of the wearer and the possibility of bacterial infection.

In addition to the absorbent products and liners of the prior art, there are currently some washable undergarment products on the market that are designed to wick moisture away from the body. These products utilize synthetic fibers to achieve their wicking properties, and generally do not offer any sort of protection against leakage to outer clothing. Doctors broadly recommend undergarments made from natural fibers (such as cotton undergarments) due to their superior breathability and hypoallergenic nature. Because of the tendency of synthetic fabrics to cause irritation and infection, many people cannot wear synthetics, especially in undergarments, and must have natural fabrics against the skin.

Thus, there remains a need for an integrated washable, low-profile, low-coverage if desired, breathable, quick-drying, odor-eliminating protective garments constructed from natural fabric and capable of capturing small amounts of bodily fluids excreted or secreted from the body so that the wearer feels dry and the wearer's other clothing is protected.

### SUMMARY OF THE INVENTION

Preferred aspects of the present invention solve the above-described problem and/or other problems by providing a novel integrated garment for capturing bodily fluids, and in particular athletic apparel configured with a novel integrated gusset or insert configured to capture and retain bodily fluids, and preferably uncontrolled urine released during athletic stress. Generally, aspects of the present invention provide a garment, and preferably a garment designed for athletic activities having a low profile and a free-range of movement against the user's body, that utilizes a fluid capture insert sewn into or onto the main body of the garment that is configured to receive small amounts of bodily fluids excreted from the wearer of the garment, including urine, menstrual fluid, vaginal discharge, sweat, breast milk, saliva, gastroesophageal reflux, blood, pus, etc. Where the bodily fluid is excreted into the interior of the garment, as is the case, for example, where the bodily fluid is urine and the garment is underwear, the capturing panel is sewn into the interior crotch portion of the garment such that it can receive the urine that is excreted.

Additional aspects of the invention may include one or more of the following preferred embodiments:

A system to manage athletic stress induced incontinence comprising:
an integrated fluid capture insert configured to be secured to a base garment structure, or a support layer of said base garment structure, wherein said integrated fluid capture insert further comprises: at least one moisture wicking layer configured to receive one or more bodily fluids from a user and draw said fluids through said wicking layer;
at least one inner absorption layer positioned adjacent to said moisture wicking layer, and further configured to absorb and retain said one or more bodily fluids; and
at least one waterproof barrier positioned adjacent to said inner absorption layer, and further configured to prevent transmission of said one or more bodily fluids from inner absorption layer to said base garment structure, or a support layer of said base garment structure.

2. The system of embodiment 1, wherein said integrated fluid capture insert is configured to be positioned within the crotch of a low profile base garment structure, or a support layer of a low profile base garment structure.

3. The system of embodiment 2, wherein said low profile base garment structure comprises athletic leggings, or shorts.

4. The system of embodiment 1, wherein said support layer of a low profile base garment structure comprises a mesh support layer.

5. The system of embodiment 1, wherein said one or more bodily fluids comprises urine expelled from a user during exercise.

6. The system of embodiment 1, wherein said moisture wicking layer comprises a moisture wicking layer made from trans-dry one-way wicking cotton.

7. The system of embodiment 1, wherein said inner absorption layer comprises an inner absorption layer made from one or more natural plant fibers, or absorbent cotton.

8. The system of embodiment 1, wherein said waterproof barrier comprises a waterproof barrier made from a non-permeable synthetic fabric.

9. The system of embodiment 1, wherein said integrated fluid capture insert configured to be secured to a base garment structure, or a support layer of said base garment structure by one or more stiches.

10. An integrated fluid capture insert comprising:
at least one moisture wicking layer configured to receive one or more bodily fluids from a user and draw said fluids through said wicking layer;
at least one inner absorption layer positioned adjacent to said moisture wicking layer, and further configured to absorb and retain said one or more bodily fluids;
at least one waterproof barrier positioned adjacent to said inner absorption layer, and further configured to prevent transmission of said one or more bodily fluids from inner absorption layer to said base garment structure, or a support layer of said base garment structure; and
wherein said integrated fluid capture insert is coupled with an article of clothing and positioned relative to a user to capture said one or more bodily fluids.

11. The insert of embodiment 10, wherein said article of clothing is selected from the group consisting of: shorts, a shirt, a bra, an athletic bra, underwear, yoga pants, athletic pants, a support layer, and a mesh support layer.

12. The insert of embodiment 10, wherein said integrated fluid capture insert is coupled with an article of clothing by mechanical stiches, a coupler mechanism, a pre-formed pocket, an adhesive, or Velcro®.

13. The system of embodiment 10, wherein said moisture wicking layer comprises a moisture wicking layer made from trans-dry one-way wicking cotton.

14. The system of embodiment 10, wherein said inner absorption layer comprises an inner absorption layer made from one or more natural plant fibers, or absorbent cotton.

15. The system of embodiment 10, wherein said waterproof barrier comprises a waterproof barrier made from a non-permeable synthetic fabric.

16. The system of embodiment 10, wherein said integrated fluid capture insert configured to be secured to a base garment structure, or a support layer of said base garment structure by one or more stiches.

17. The insert of embodiment 10, wherein said one or more bodily fluids comprises urine expelled from a user during exercise.

18. The insert of embodiment 10, wherein said inner absorption layer comprises an inner absorption layer having variable amounts of absorption material according to a user's need.

19. The insert of embodiment 10, wherein said article of clothing comprises an article of clothing made from a fabric selected from the group consisting of: a washable fabric, a fabric created from natural or synthetic fibers, a textured fabric, a treated fabric, or a combination of the same.

20. A low profile athletic legging configured to manage athletic stress induced incontinence comprising:
an integrated fluid capture insert configured to be secured to a mesh support layer of a low profile athletic leggings, wherein said integrated fluid capture insert further comprises:
at least one moisture wicking layer configured to receive urine involuntarily expelled from a user during exercise and draw the urine through said wicking layer;
at least one inner absorption layer positioned adjacent to said moisture wicking layer, and further configured to absorb and retain said urine involuntarily expelled from a user during exercise; and
at least one waterproof barrier positioned adjacent to said inner absorption layer, and further configured to prevent transmission of said urine involuntarily expelled from a user during exercise from inner absorption layer to said mesh support layer, or low profile athletic leggings.
wherein said integrated fluid capture insert is positioned within the crotch of said mesh support layer such that said urine involuntarily expelled from a user during exercise is directed into the insert.

Additional aspects of the invention may become evident from the detailed description and figures generally described below.

### BRIEF DESCRIPTION OF THE FIGURES

Aspects, features, and advantages of the present disclosure will be better understood from the following detailed descriptions taken in conjunction with the accompanying figures, all of which are given by way of illustration only, and are not limiting the presently disclosed embodiments, in which:

FIG. 1: shows a cross section of a fluid capture insert secured to an exemplary garment fabric in one embodiment thereof;

FIG. 2: shows a top blow-up view of the individual layer components of a fluid capture insert secured to the crotch of an exemplary garment fabric in one embodiment thereof;

FIG. 3: shows a top unexpanded view of a fluid capture insert secured to the crotch of an exemplary garment fabric in one embodiment thereof; and

FIG. 4: shows an integrated fluid capture insert (1) integrated with low profile athletic legging in one embodiment thereof.

### DETAILED DESCRIPTION OF THE INVENTION

The present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

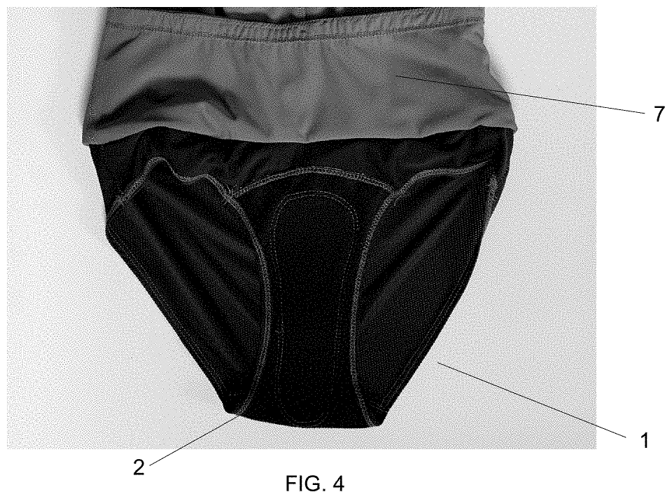

In one embodiment, the invention may include a base garment structure (7) may be provided that may include a novel fluid capture insert (1). The underlying base garment structure (7) of the invention may be any piece of wearable clothing, and preferably athletic apparel which may encompass low profile garments configured to accommodate athletic movement. For example, as shown in FIG. 4, in one preferred embodiment, the base garment structure (7) of the invention may include low profile leggings that may be particularly suited for running or other physical activities. Additional embodiment of the invention may include a base garment structure (7) may be provided that may include a novel fluid capture insert (1), wherein the garment selected from the group consisting of: shorts, a shirt, a bra, an athletic bra, underwear, pants, and preferably yoga or athletic pants and the like. In some embodiments, the base garment structure (7) of the invention may include a low-to-moderate coverage garment that is appropriate for daily wear and may further include base garment structures (7) generally designed for both men, women, and unisex. It should be understood that the foregoing examples of base garment structures (7) are not exhaustive of the form that the base garment structures (7) may take in accordance with different embodiments of the present invention.

The present invention may include a fluid capture insert (1) that may be secured to a garment in a position to capture one or more bodily fluids. The fluid capture insert (1) of the invention may include a plurality or layers that may be coupled together and positioned strategically on a garment to capture one or more bodily fluids. The fluid capture insert (1) may be integrated into a garment during the manufacturing process, or may be separately secured to the garment, for example by a coupler mechanism, such as an adhesive, Velcro® or snap coupler and the like. In this configuration, the fluid capture insert (1) may be modular such that a user may adjust the size, or number of inserts in the garment, for example based on the anticipate intensity or length of a planned exercise activity. This modular embodiment further allows the user to quickly replace the fluid capture insert (1) as it becomes saturated, or near-saturated with bodily fluid.

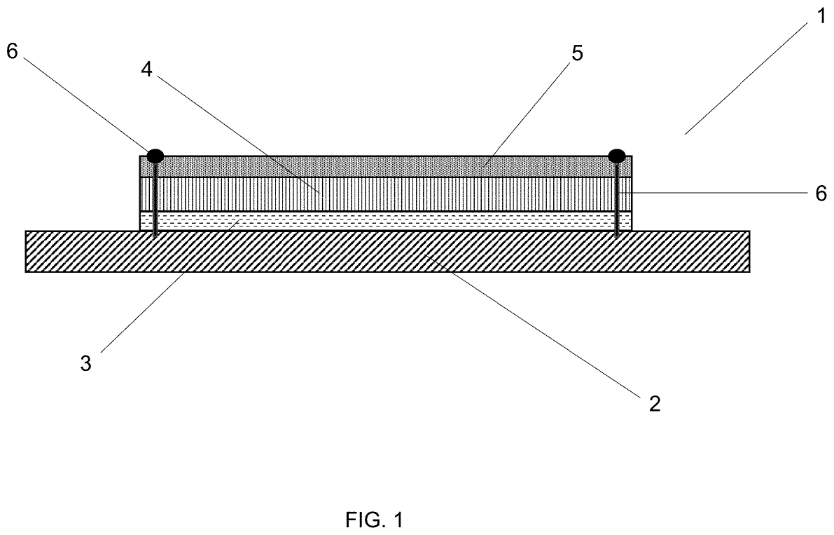

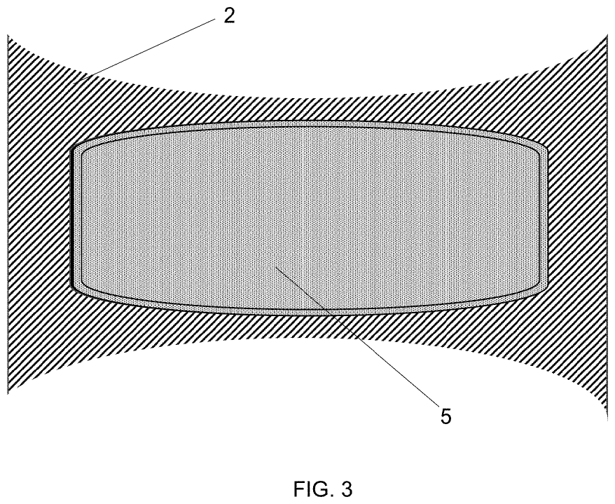

Referring to FIG. 1, fluid capture insert (1) may include a plurality of individual layers positioned adjacent to one another forming a unitary component that may work synergistically to wick bodily fluids away from a user's body and into the insert's internal layers to be collected, while having an outer fluid barrier to prevent captured fluid from flowing through to the inserts outer surface layer. Specifically, in the embodiment shown in FIGS. 1-4, a base garment structure (7) may include a support brief. A support brief may be formed by a separate layer from an underlying garment and may be further shaped to fit a user's body in a low profile configuration. As specifically shown in FIG. 4, a base garment structure (7) may include a garment structure similar to underwear that may be positioned tightly around a user's upper legs and crotch. As noted above, additional embodiment may include base garment structure (7) that may be configured to secure a fluid capture insert (1) may include, for example a shirt, bra, short and the like. Moreover, base garment structure (7) may be constructed from a single piece of fabric or may be constructed from multiple pieces of material that are sewn or otherwise attached to each other. According to different embodiments, the material used to construct the base garment structure (7) may be any type of washable fabric, including fabrics created from natural or synthetic fibers, textured fabric, including lace, treated fabric, a combination of the foregoing, etc.

By way of example, embodiments that include athletic leggings may be made of a polyester/spandex blend. For example, in this preferred embodiment, the athletic leggings may include a mixture of polyester/spandex, as an 88% and 12% mix, respectively. Additional example athletic leggings having a higher degree of compression may include a mixture of polyester/spandex 76% of 24%, respectively. Yet further embodiment may include, athletic leggings may have a mixture of cotton and spandex, for example a mixture of 93% and 7% respectively.

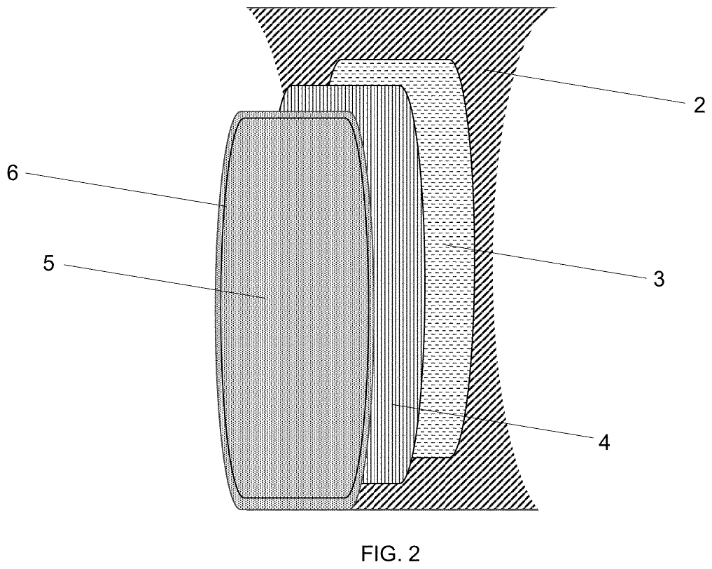

According to some embodiments, the fluid capture insert (1) is constructed out of washable fabric material and is configured to receive and retain bodily fluid excreted by a wearer of the base garment structure (7). Referring to FIGS. 1-2, in a preferred embodiment a fluid capture insert (1) may include moisture wicking layer (5) positioned adjacent to a user's body that is configured to wick away a quantity of bodily fluids that may be expressed or leaked from a user. In a preferred embodiment, moisture wicking layer (5) may include any appropriate moisture-wicking fabrics configured to pull moisture away from a user's skin, for example using small integrated capillaries structures that draw moisture through a series of capillary-action driven movements to the exterior of the fabric making it easier to evaporate, or be passed to an adjacent absorption layer. In one preferred embodiment, the moisture wicking layer (5) of a fluid capture insert (1) may include a trans-dry one-way wicking cotton, such as Zorb® fabric produced by Wazoodle Fabrics™.

Referring to FIGS. 1-2, in a preferred embodiment a fluid capture insert (1) may include an inner absorption layer (4) positioned below a moisture wicking layer (5) as generally described above. In this preferred embodiment, the inner absorption layer (4) may be configured to absorb and hold a quantity of bodily fluids that may be expressed or leaked from a user that is passed through by the wicking layer (5). The inner absorption layer (4) may be configured to have a variable quantity such that the amount of bodily fluid, such as urine, that may be absorbed prior to saturation by the inner absorption layer (4) may be customizable based on a user's desire or need. In one preferred embodiment, the inner absorption layer (4) may be formed by a quantity of natural plant fibers, such as bamboo, or absorbent cotton and the like.

Referring to FIGS. 1-2, in a preferred embodiment a fluid capture insert (1) may further include waterproof barrier (3) positioned below the inner absorption layer (4). In this preferred embodiment, the waterproof barrier (3) may be configured to form a non-permeable barrier to prevent bodily fluid, such as urine, absorbed and held by the inner absorption layer (4) from passing through to the outside of the base garment structure (7).

According to different embodiments, the waterproof barrier (3) may be formed by a non-permeable synthetic fabric, such as a polyester. In other embodiment, the waterproof barrier (3) may be generated by application of a laminate, coating, or durable water repellant treatment to the fabric waterproof barrier (3). For example, according to different embodiments, the fluid-resistant treatment may include a porous fluoropolymer membrane coating or a finishing treatment. The fluid-resistant treatment may be applied through a bath or by spray-on or foam applications. According to one embodiment, the fluid-resistant treatment comprises a durable water repellant treatment that includes subjecting the fabric of waterproof barrier (3) to a nanoparticle C6 fluorochemical finishing bath. The nanoparticles form a fine structure on the surface of the fabric that prevents fluid and other substances from adhering. Fabric treated in this manner will not only be fluid-resistant, but as an added advantage, will also be resistant to stains. Further, the treated fabric is breathable and will maintain its resistance to fluid even after multiple washings of the base garment structure (7).

As generally shown in FIGS. 1-4, in a preferred embodiment, the above described layers of the fluid capture insert (1) may be integrated into a support layer (2), such as a mesh support layer configured to be secured to a user like underwear. In addition embodiment, the fluid capture insert (1) may be integrated into a support layer (2) by positioning the layer one on top of the other and securing them directly to a support layer (2) or base garment structure (7), for example through one or more manual or machine stiches (6). As noted above, additional embodiments, the fluid capture insert (1) may be secured to a support layer (2) or base garment structure (7) in a modular fashion, such as through an adhesive, Velcro®, or other similar low-profile coupler device.

It should be noted that the figure in 1-4 demonstrate one preferred shape of a fluid capture insert (1) that may be configured to be placed within the crotch of the support layer (2) or base garment structure (7) and further configured to capture and retain bodily fluids, and preferably uncontrolled urine released by a female during athletic stress, however, additional shapes and configuration are contemplated within the scope of this invention. For example, a fluid capture insert (1) may be positioned to capture and retain bodily fluids, and preferably uncontrolled urine released by a male during athletic stress, while additional embodiments may be positioned within a bra or shirt and configured to capture, for example involuntary expressed breast milk, sweat and the like.

Naturally, all embodiments discussed herein are merely illustrative and should not be construed to limit the scope of the inventive technology consistent with the broader inventive principles disclosed. As may be easily understood from the foregoing, the basic concepts of the present inventive technology may be embodied in a variety of ways. It generally involves systems, methods, techniques as well as devices to accomplish a novel integrated gusset for athletic apparel and the like. In this application, the methods and apparatus for the aforementioned systems are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention. As can be easily understood from the foregoing, the basic concepts of the present invention may be embodied in a variety of ways. It involves both techniques as well as devices to accomplish the appropriate system. In this application, the techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in method-oriented terminology, each element of the claims corresponds to a device. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “support” should be understood to encompass disclosure of the act of “supporting”—whether explicitly discussed or not—and, conversely, were there effectively disclosure of the act of “supporting”, such a disclosure should be understood to encompass disclosure of a “supporting method and/or technique, and/or device” and even a “means for supporting.” Such changes and alternative terms are to be understood to be explicitly included in the description.

Any patents, publications, or other references mentioned in this application for patent, such as in the specification or an IDS are hereby incorporated herein by reference in their entirety. Any priority case(s) claimed by this application is hereby appended and hereby incorporated herein by reference in their entirety. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated herein by reference in their entirety. Finally, all references listed in the list of References To Be Incorporated By Reference In Accordance With The Patent Application or other information disclosure statement and the like filed with the application are hereby appended and hereby incorporated herein by reference in their entirety, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the methods and/or apparatus for providing a novel integrated gusset for athletic apparel as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. It should be understood that this application also provides support for any combination of elements in the claims and even incorporates any desired proper antecedent basis for certain claim combinations such as with combinations of method, apparatus, process, and the like claims.

Any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The inventive subject matter is to include, but certainly not be limited as, a system substantially as herein described with reference to any one or more of the Figures and Description (including the following: for example, the process according to any claims and further comprising any of the steps as shown in any Figures, separately, in any combination or permutation).

What is claimed is:

1. A system to manage athletic stress induced incontinence comprising:
an integrated fluid capture insert configured to be secured to a base garment structure, or a support layer of said base garment structure, wherein said integrated fluid capture insert further comprises:
at least one moisture wicking layer configured to receive one or more bodily fluids from a user and draw said fluids through said wicking layer;
at least one inner absorption layer positioned adjacent to said moisture wicking layer, and further configured to absorb and retain said one or more bodily fluids; and
at least one waterproof barrier positioned adjacent to said inner absorption layer, and further configured to prevent transmission of said one or more bodily fluids from inner absorption layer to said base garment structure, or a support layer of said base garment structure.

2. The system of claim 1, wherein said integrated fluid capture insert is configured to be positioned within the crotch of a low profile base garment structure, or a support layer of a low profile base garment structure.

3. The system of claim 2, wherein said low profile base garment structure comprises athletic leggings, or shorts.

4. The system of claim 1, wherein said support layer of a low profile base garment structure comprises a mesh support layer.

5. The system of claim 1, wherein said one or more bodily fluids comprises urine expelled from a user during exercise.

6. The system of claim 1, wherein said moisture wicking layer comprises a moisture wicking layer made from trans-dry one-way wicking cotton.

7. The system of claim 1, wherein said inner absorption layer comprises an inner absorption layer made from one or more natural plant fibers, or absorbent cotton.

8. The system of claim 1, wherein said waterproof barrier comprises a waterproof barrier made from a non-permeable synthetic fabric.

9. The system of claim 1, wherein said integrated fluid capture insert configured to be secured to a base garment structure, or a support layer of said base garment structure by one or more stiches.

10. An integrated fluid capture insert comprising:
at least one moisture wicking layer configured to receive one or more bodily fluids from a user and draw said fluids through said wicking layer;
at least one inner absorption layer positioned adjacent to said moisture wicking layer, and further configured to absorb and retain said one or more bodily fluids;
at least one waterproof barrier positioned adjacent to said inner absorption layer, and further configured to prevent transmission of said one or more bodily fluids from inner absorption layer to said base garment structure, or a support layer of said base garment structure; and
wherein said integrated fluid capture insert is coupled with an article of clothing and positioned relative to a user to capture said one or more bodily fluids.

11. The insert of claim 10, wherein said article of clothing is selected from the group consisting of: shorts, a shirt, a bra, an athletic bra, underwear, yoga pants, athletic pants, a support layer, and a mesh support layer.

12. The insert of claim 10, wherein said integrated fluid capture insert is coupled with an article of clothing by mechanical stiches, a coupler mechanism, a pre-formed pocket, an adhesive, or Velcro®.

13. The system of claim 10, wherein said moisture wicking layer comprises a moisture wicking layer made from trans-dry one-way wicking cotton.

14. The system of claim 10, wherein said inner absorption layer comprises an inner absorption layer made from one or more natural plant fibers, or absorbent cotton.

15. The system of claim 10, wherein said waterproof barrier comprises a waterproof barrier made from a non-permeable synthetic fabric.

16. The system of claim 10, wherein said integrated fluid capture insert configured to be secured to a base garment structure, or a support layer of said base garment structure by one or more stiches.

17. The insert of claim 10, wherein said one or more bodily fluids comprises urine expelled from a user during exercise.

18. The insert of claim 10, wherein said inner absorption layer comprises an inner absorption layer having variable amounts of absorption material according to a user's need.

19. The insert of claim 10, wherein said article of clothing comprises an article of clothing made from a fabric selected from the group consisting of: a washable fabric, a fabric created from natural or synthetic fibers, a textured fabric, a treated fabric, or a combination of the same.

20. A low profile athletic legging configured to manage athletic stress induced incontinence comprising:
an integrated fluid capture insert configured to be secured to a mesh support layer of a low profile athletic leggings, wherein said integrated fluid capture insert further comprises:
at least one moisture wicking layer configured to receive urine involuntarily expelled from a user during exercise and draw the urine through said wicking layer;
at least one inner absorption layer positioned adjacent to said moisture wicking layer, and further configured to absorb and retain said urine involuntarily expelled from a user during exercise; and
at least one waterproof barrier positioned adjacent to said inner absorption layer, and further configured to prevent transmission of said urine involuntarily expelled from a user during exercise from inner absorption layer to said mesh support layer, or low profile athletic leggings.
wherein said integrated fluid capture insert is positioned within the crotch of said mesh support layer such that said urine involuntarily expelled from a user during exercise is directed into the insert.
